(19) United States
(12) Patent Application Publication
(10) Pub. No.: US 2023/0001356 A1
(43) Pub. Date: Jan. 5, 2023

(54) IN SITU FILTRATION FOR A BIOCONTAINER

(71) Applicant: EMD Millipore Corporation, Burlington, MA (US)

(72) Inventors: Stefano Berti Perez, Boston, MA (US); Alison Dupont, Lyndeborough, NH (US)

(21) Appl. No.: 17/772,724

(22) PCT Filed: Nov. 17, 2020
(86) PCT No.: PCT/US2020/060832

§ 371 Date: Apr. 28, 2022

Related U.S. Application Data
(60) Provisional application No. 62/963,704, filed on Jan. 21, 2020.

(51) Int. Cl. B01D 61/18; C12M 1/00

(52) U.S. Cl. CPC B01D 61/18; C12M 47/10; C12M 29/04; B01D 2313/20; B01D 2313/58

(57) ABSTRACT

The disclosure herein relates to a TFF assembly including a baffle comprising a microporous filter within a bioreactor. The baffle may include a collection receptacle to capture a target product or waste materials within the baffle after passing through the microporous filter.

### RELATED APPLICATIONS

The present application claims the benefit of priority U.S. Priority Patent Application No. 62/963,704, filed Jan. 21, 2020, the entire contents of which is incorporated herein in its entirety.

### FIELD OF THE DISCLOSURE

Embodiments disclosed herein relate to bioprocessing. More specifically, embodiments of the technology relate to a tangential flow filtration (TFF) assembly within a bioreactor. In some embodiments, the TFF assembly also functions as a baffle during bioprocessing within the bioreactor.

### BACKGROUND OF THE DISCLOSURE

Perfusion systems and processes involve filtration of fluid within a bioreactor during semi- and/or continuous bioprocessing. During filtration, target products or other soluble components, such as cellular waste materials (e.g., lactic acid and ammonia), are removed from the bioreactor. Perfusion processes rely on a high density of host cells being maintained throughout each production process, and continuous harvesting, which involves several iterations of filtration, potentially causing physical damage to the host cells. Additionally, conventional perfusion systems and processes use filter elements having open feed channels to avoid obstructions that could damage host cells. Filter elements used in such conventional systems and processes result in relatively low viability and exhibit significantly reduced sieving at low harvest throughputs due to membrane fouling.

Use of cylindrical bioreactor tanks or bags result in vortex formation during bioprocessing regardless of the type of impeller utilized for mixing. Vortices result in decreased mixing and stagnant zones. Baffles are placed within the reusable or single-use bioreactors disclosed herein to prevent or disrupt vortex formation and enhance fluid movement to improve mixing by delivering fluid into a more desirable flow pattern that includes both axial and radial flow. Baffles have also been used with dynamic membrane bioreactors.

The position of the vortex changes with aspect ratio. The region where the vortex would form in the absence of the baffle can be determined from experience or by mixing fluid in the inner volume under similar mixing conditions that will be used in operation, but in the absence of the baffle, and noting where the vortex forms. A “vortex map” can be created, documenting the location of the vortex for a given bioreactor aspect ratio, volume, mixer position, and mixer size.

Tangential flow filtration (TFF) is a separation process that uses membranes to separate components in a liquid solution or suspension on the basis of size or molecule weight differences. In conventional TFF, the solution or suspension to be filtered is passed across the surface of the membrane in a cross-flow mode, i.e., tangential to a membrane surface. The velocity at which the filtrate is passed across the membrane surface also controls the filtration rate and helps prevent clogging of the membrane. TFF is used frequently in perfusion systems to remove target proteins from a bioreactor during cell culturing, while retaining cells in a bioreactor for further production. TFF recirculates retentate across the membrane surface to reduce membrane fouling, maintain a high filtration rate, and enhance product recovery compared to other filtration methods. TFF also provides lesser shear rates compared with other filtration processes.

Applications of TFF include the concentration of biological product(s); clarification and desalting of proteins and other biomolecules from a solution or suspension, such as nucleotides, antigens, and monoclonal antibodies; pre-chromatographic clarification to remove colloidal particles; depyrogenation of small molecules, such as dextrose and antibiotics; harvesting; washing or clarification of cell cultures, lysates, colloidal suspensions, and viral cultures; and sample preparation.

Conventional TFF devices are formed of a plurality of elements, including a pump, a feed solution reservoir, a filtration module and conduits for connecting these elements. In use, the feed solution is directed from the feed solution reservoir to the filtration module, while the retentate from the filtration module is recirculated from the filtration module to the feed solution reservoir until the desired volume of retentate is obtained. In the traditional TFF devices, the membrane is sandwiched between top and bottom manifolds or holders, which provide accurate mechanical constraint against the internal hydraulic pressure of the device. Some previous bioreactors also have included stationary filtration devices within the bioreactor with a mixing assembly able to rotate around the filtration device.

Current TFF devices used in perfusion systems include hollow fiber devices and open-channel cassette devices, also referred to as plate-and-frame devices. Examples of commercialized cassette devices for perfusion systems include XCell™ ATF System (Repligen, Waltham, Mass.) and KrosFlo® Perfusion System (Spectrum Laboratories, Rancho Dominguez, Calif.), which are hollow fiber devices, and Prostak™ Microfiltration Modules (MilliporeSigma, Burlington, Mass.).

The TFF devices presently used in the art require high cross-flow rates to minimize fouling (i.e., the accumulation of particles along the wall of membrane). Eventually, membrane fouling can result in failure of the device, with product no longer being recovered during filtration. In current perfusion systems or processes, the desired cross-flow rate is achieved by an external pump in fluid communication, such as being welded or glued, with the bioreactor to move the cells within the bioreactor to an external device containing the membrane. Then, the permeate is pumped back into the bioreactor where bioprocessing is occurring. Removing and reintroducing the permeate into a perfusion system or process increases the chances of contamination compared to systems that retain the permeate in a single bioreactor.

A TFF assembly for use in a bioreactor that does not need a pump, which reduces shear to minimize cell damage, and reduces the likelihood of contamination for ongoing production of a target product during perfusion bioprocessing represents an inventive advance in the art.

### SUMMARY OF THE DISCLOSURE

The shortcomings of the prior art are overcome by embodiments described herein, which include various embodiments disclosed herein providing a bioreactor containing a TFF device; having a baffle supporting a membrane. In some embodiments, the membrane is a microporous membrane.

Various embodiments disclosed herein include a TFF assembly comprising: a microporous membrane; and a baffle supporting the microporous membrane.

In some embodiments, the TFF assembly may be a flat plate. In some embodiments, the TFF assembly may be shaped as at least one shape selected from the group consisting of: a rectangle, a trapezoid, a parallelogram, a circle, an ellipse, a racetrack, a triangle, and a ladder. In some embodiments, the TFF assembly further comprises a collection receptacle sealed to the baffle. In some embodiments, the TFF assembly further comprises an outlet on the collection receptacle.

Some embodiments disclosed herein comprise a bioreactor having: an inner volume enclosed by at least one side wall; a TFF assembly including a baffle supporting a microporous membrane, wherein the filtration assembly is movably attached to the side wall a mixer within the inner volume.

In some embodiments, the bioreactor comprises one or more inlets and/or one or more outlets. In some embodiments, the baffle spans the height of the inner volume. In some embodiments, the baffle spans enough of the radial dimension of the inner volume to disrupt vortex formation. In some embodiments, the baffle is a flat plate. In some embodiments, the baffle is shaped as at least one shape selected from the group consisting of: a rectangle, a trapezoid, a parallelogram, a circle, an ellipse, a racetrack, a triangle, and a ladder.

In some embodiments, the bioreactor is single use and/or disposable. In some embodiments, the bioreactor further comprises more than one TFF assembly. In some embodiments, the bioreactor is a perfusion bioreactor. In some embodiments, the bioreactor is collapsible. In some embodiments, the bioreactor comprises a flexible material. In some embodiments, the TFF assembly is a flexible film. In some embodiments, more than one TFF assembly is attached to a side wall of the inner volume. In some embodiments, the bioreactor does not include an external pump. In some embodiments, the bioreactor does not include a feed line. In some embodiments, the bioreactor is self-contained. In some embodiments, the bioreactor further comprises a collection receptacle sealed to the baffle. In some embodiments, the bioreactor further comprises an outlet on the collection receptacle. In some embodiments, the bioreactor further comprises an outlet on the collection receptacle. In some embodiments, the bioreactor further comprises an external pump.

Some embodiments disclosed herein include a method of removing at least one product or waste material out of a bioreactor, the method comprising: performing perfusion bioprocess in the bioreactor described herein. In some embodiments, bioprocessing includes at least one selected from the group consisting of cell bioprocessing, cell culture, diafiltration, and downstream bioprocessing.

In some embodiments, the method further comprises at least semi-continuous sweeping of the microporous membrane by mixing contents within the bioreactor. In some embodiments, the method further comprises breaking, preventing, or minimizing vortex formation within the bioreactor. In some embodiments, the method described herein further comprises maintaining a homogenous mixture of the contents within the bioreactor. In some embodiments, the product is a therapeutic modality. In some embodiments, the contents within the bioreactor comprise microcarriers. In some embodiments, the method described herein further comprises preventing contamination by reducing the number of containers the contents within the bioreactor are transferred during perfusion cell culture. In some embodiments of the method described herein, removing comprises collecting the product or the waste material in the collection receptacle. In some embodiments, cell culture comprises cultivating at least one type of cell selected from the group consisting of: plant, animal, fungus, bacteria, and hybridoma cell line. For example, the hybridoma cell line may be selected from a Chinese hamster ovary (CHO) cell line or a NS0 (murine myeloma) cell line.

### BRIEF DESCRIPTION OF THE DRAWINGS

FIG. 1A is an upper perspective view of some embodiments of the TFF assembly described herein. FIG. 1B is a front view of some embodiments of the TFF assembly described herein. FIG. 1C is a top view of some embodiments of the TFF assembly described herein.

FIG. 2A is a side view of some embodiments of the bioreactor described herein. FIG. 2B is an upper perspective view of some embodiments of the bioreactor described herein.

The appended drawings illustrate some embodiments of the disclosure herein and are therefore not to be considered limiting in scope, for the invention may admit to other equally effective embodiments. It is to be understood that elements and features of any embodiment may be found in other embodiments without further recitation and that, where possible, identical reference numerals have been used to indicate comparable elements that are common to the figures.

### DETAILED DESCRIPTION OF THE DISCLOSURE

The disclosure herein describes some embodiments of a TFF assembly to improve the performance of bioprocessing systems and processes. Some embodiments disclosed herein result in a homogeneous mixing state provided by the baffle and can provide even distribution of support matrices for adherent cells, such as microcarriers, increasing potential for good growth of certain cells, including, but not limited to stem cells. An effective mixing system provides three basic functions: creation of constant conditions (nutrients, pH, temperature, etc.) in a homogeneous distribution; dispersion of gas, e.g., oxygen; and extracting carbon dioxide where and when needed as in a bioreactor; and optimization of heat transfer.

Generally, the pump used during perfusion bioprocessing exacts a lot of shear. The reusable and single-use bioreactors disclosed herein reduce shear during mixing the fluid in a bioreactor because overall power input can be reduced while still maintaining good mixing. Lower power input translates to lower shear. With more homogeneous mixing at lower power input, a larger process window for cell culture processes is provided, providing greater flexibility in finding optimum process conditions to eliminate shear.

Locating the TFF assembly within the bioreactor as described herein also reduces the physical footprint of the bioreactor and self-containment decreases the likelihood of contamination. Minimizing the number of containers into which bioreactor contents need to be transferred, since each transfer represents a potential breach of sterility, and, frequently, the resulting contamination cannot be filtered away, is favorable. For example, it would be beneficial to mix vaccines in the same bioreactor, such as a flexible, disposable bag, that the vaccines will be shipped within because liquids in vaccines often contain aluminum salt as an adjuvant, which improves the efficacy of the vaccine by enhancing the body's immune response. The aluminum salts consist of particle sizes larger than 0.2 μm, thus sterile filtering generally is not an option. Due to limitations of space in most laboratories, minimal space requirements and small footprints are also a long felt need in the field.

### I. Tangential Flow Filtration (TFF)

The TFF assembly describes herein provides for at least semi-continuous sweeping of the contents of a bioreactor as long as the contents are being mixed and flowing over the microporous filter supported by a baffle. In some embodiments, the target product may be captured in a collection receptacle attached to or which is part of the TFF assembly. In further embodiments, the present disclosure includes recovering the target product in the permeate and/or retaining waste media in the retentate. Alternatively, the present disclosure also includes recovering waste material(s) in the permeate and/or retaining the target product in the retentate.

Some embodiments herein describe a TFF assembly, which also functions as a baffle while a target product is being produced within the bioreactor. The TFF assembly may be used to remove waste or impurities during perfusion cell culture. Alternatively, the TFF assembly may be used to capture a target product during perfusion cell culture. Cell culture may be performed for any type of cells, including: plant, animal (e.g., insect), bacteria, fungus (e.g., yeast), and hybridoma cells, which can be grown in a cell culture medium. For example, a cultivated cell line is a Chinese hamster ovary (CHO) or NS0 cell line (murine myeloma cells). The target product may be produced by microbiological applications, such as cultivating microorganisms, specifically, bacteria, or fungi, e.g., yeast.

### II. Bioreactor Design

Embodiments disclosed herein include reusable and disposable or single-use bioreactors, optionally having one or more inlets and one or more outlets and a mixer associated with the inner volume of the bioreactor to cause mixing, dispersing, homogenizing, and/or circulation of one or more ingredients contained or added to the inner volume.

The bioreactors described herein are envisioned to hold volumes of up to 10 L or more, specifically with a total volume of approximately 0.35, 1.5, 5.0, 10 L with a working volume ranging between about 700 and 1300 ml, about 1 to 3 L, or about 2.5 to 10 L. In some embodiments, the bioreactor holds a volume of up to about 100 L, about 200 L, about 500 L, about 1000 L, about 2000 L, about 2500 L, or about 3000 L.

In some embodiments, the bioreactor has a temperature control unit to maintain the fluid associated with bioprocessing, e.g., cell culture, at a consistent temperature.

### A. Inner Volume

In accordance with some embodiments, the bioreactor disclosed herein is a disposable container designed to receive and hold a fluid. The bioreactor encloses a space referred to as an inner volume in which biological or biotechnological processes can be carried out on a laboratory scale. Such processes include the cultivation of cells, microorganisms, or small plants under defined, controlled, and reproducible conditions.

In some embodiments, the bioreactor comprises or consists of a material conforming to the United States Pharmacopeia (USP) Class VI requirements, such as a plastic material. The plastic material may be polyamide, polycarbonate, polymethylpentene, or polystyrene. The disposable bioreactor may be formed of monolayer or multilayer flexible walls of a polymeric composition such as polyethylene, for example, ultra-high molecular weight polyethylene, linear low density polyethylene, low density or medium density polyethylene, polypropylene, ethylene vinyl acetate (EVOH), polyvinyl chloride (PVC), polyvinyl acetate (PVA), ethylene vinyl acetate copolymers (EVA copolymers), blends of various thermoplastics, co-extrusions of different thermoplastics, multilayered laminates of different thermoplastics, or the like as described in US20190210321 and WO2019199406, which are hereby incorporated by reference in entirety. “Different” is meant to include different polymer types such as polyethylene layers with one or more layers of EVOH as well as the same polymer type but of different characteristics such as molecular weight, linear or branched polymer, fillers, and the like. Typically, medical grade and preferably animal-free plastics are used, which are generally are sterilizable such as by steam, ethylene oxide, or radiation, such as beta or gamma radiation. Most have good tensile strength, low gas transfer, and are either transparent or at least translucent. In some embodiments, the material is weldable or gluable to form a fluid tight connection with other features of a bioreactor and is unsupported. In some embodiments, welding techniques can be selected from the group consisting of plastic welding or heat sealing, for example, ultrasonic welding, laser welding, welding using infra-red radiation, or thermal welding. In some embodiments, the material is clear or translucent, allowing visual monitoring of the contents. In some embodiments, the bioreactor is integrally formed in an injection molding process or a blow molding process.

In some embodiments, the bioreactor is provided with one or more inlets, one or more outlets, and/or one or more optional vent passages allowing access to the inner volume. In some embodiments, the inner volume of the bioreactor is of a sufficient size to contain fluid to be mixed, such as cells and a culture medium. In some embodiments, the inner volume of the bioreactor is capable of supporting a biologically active environment, such as one capable of growing cells in the context of cell cultures.

In some embodiments, the bioreactor is a disposable, deformable, and/or foldable bag defining an inner volume, that is sterilizable for a single use, capable of accommodating contents, such as biopharmaceutical fluids, in a fluid state, and that can accommodate a mixing device partially or completely within the inner volume. In some embodiments, the inner volume can be opened, such as by suitable valving, to introduce a fluid into the volume, and to expel fluid therefrom, such as after mixing is complete. In some embodiments, the bioreactor may be a two-dimensional or “pillow” bag, or the bioreactor may be a three-dimensional bag. The particular geometry of the bioreactor is not limited. In some embodiments, the bioreactor includes a rigid base, which provides access points to the inner volume, such as ports or vents. Each bioreactor may contain one or more inlets and outlets and optionally other features, such as sterile gas vents and ports for the sensing of the liquid of the inner volume for parameters, such as conductivity, pH, temperature, dissolved gases, and the like. In some embodiments, the bioreactor includes sensors. Such sensors typically monitor pH, dissolved gases, temperature, conductivity, and the like to determine homogeneity throughout the inner volume. To do so, sensors are often placed within dip tubes from the top of the bag into the inner volume of the bioreactor at one or more locations. Alternatively, the sensors are mounted to a wall of the inner volume.

### B. Baffle

In some embodiments, the bioreactor includes two or more baffles within the inner volume. The two or more baffles may have equal or similar dimensions. The baffle is at least partially submerged in the fluid within the inner volume to enhance disruption of the vortex across the entire vessel height and provide homogeneous mixing throughout all operating volumes. In some embodiments, the baffle is positioned in the inner volume to extend through the vortex or the region where the vortex would form in the absence of the baffle.

In accordance with some embodiments, the baffle member should be wide enough (with respect to the radial dimension of the inner volume) to disrupt the vortex formation at the surface of the fluid, but not too wide to block flow from side-to-side within the inner volume, which would increase the time for mixing the entire volume. The dimensions of the baffle depend in part on the size of the inner volume.

In some embodiments, the baffle comprises or consists of a material which conforms to United States Pharmacopeia (USP) Class VI requirements, such as a plastic material, for example, polyamide, polycarbonate, polymethylpenten, polypropylene, or polystyrene. In some embodiments, at least the outer wall of the baffle comprises or consists of the material conforming to United States Pharmacopeia (USP) Class VI requirements.

In some embodiments, the microporous membrane supported by the baffle has a mean pore size of at least about 0.65 μm, for example, 0.62, 0.65, 0.67, or 0.80 μm; at least about 1.0 μm, for example, 0.95, 1.0, or 1.2 μm; at least about 3.0 μm, for example, 2.9, 3.0, or 5.0 μm. The mean pore size of the microporous membrane may be about 0.8 μm to about 10 μm, for example, 0.77, 0.8, 0.9, 2, 4, 6, 8, or 10.3 μm; or about 1.0 to about 5 μm, for example, 0.97, 1.2, 3, or 5.3 μm. The mean pore size can be selected to provide for sieving of target products and/or waste materials from a cell culture fluid, while retaining cells within the cell culture fluid. Examples of suitable microporous membranes include the membranes listed in Table 1 of WO2018222550, which is hereby incorporated by reference in its entirety.

In some embodiments, the baffle includes at least partially hollow interior acting as a collection receptacle for a target product or waste material resulting from bioprocessing. The collection receptacle may be separated from the inner volume in a fluid tight manner. The collection receptacle may allow the fluid to remain in the baffle, or the baffle may include a fluid connector as an outlet for fluid to flow away from the collection receptacle. In some embodiments, the collection receptacle is adapted to withstand fluid pressure with a range of up to about 68 kPa to up to about 70 kPa, which is equivalent to about 10 psi. For example, the collection receptable can withstand a head pressure of the bioreactor is about 6.77 psi. In some embodiments, the collection receptable can withstand pressure associated with fouling of the TFF assembly, e.g. an additional 3 psi of pressure.

The fluid connector may be made of materials with United States Pharmacopeia (USP) Class VI certification, such as polystyrene, polycarbonate, polyamide, or silicone. In some embodiments, the fluid connector is a flexible tube made of thermal plastic elastomers. Alternatively, rigid tubes comprising or consisting of polystyrene, polycarbonate, or polyamide may be used as a fluid connector. In some embodiments, the fluid connector is attached to a pump. In some embodiments, the fluid connector is arranged with the collection receptacle or pump coaxially.

### C. Mixer

In some embodiments, each inner volume contains, either partially or completely within its interior, a mixer for mixing, dispersing, homogenizing, and/or circulating one or more liquids, gases, and/or solids in the inner volume. In some embodiments, the mixer may include one or more blades, which are movable, such as by rotation or oscillation about an axis. In some embodiments, the mixer includes a shaft rotated by a drive, as a result the blades also rotate to mix the fluid and/or solids in the inner volume. Alternatively, the mixer is magnetically coupled to the motor so no shaft penetrates the bioreactor. In some embodiments, the baffle is not attached or contacted to the mixer. In some embodiments, the baffle rotates or pivots separately from the mixer.

The mixer may have a protective hood formed over at least a part of the blades with a space contained between the under surface of the hood and the outer dimension of the blades so as to allow for free movement of the blades and fluid between the blades and the under surface of the hood. The hood protects the wall of the inner volume from the blades that could otherwise damage the bioreactor.

More than one mixer may be enclosed by the bioreactor. When more than one mixer is present, each is spatially arranged to not interfere with the rotation of the other.

### III. Bioprocessing System

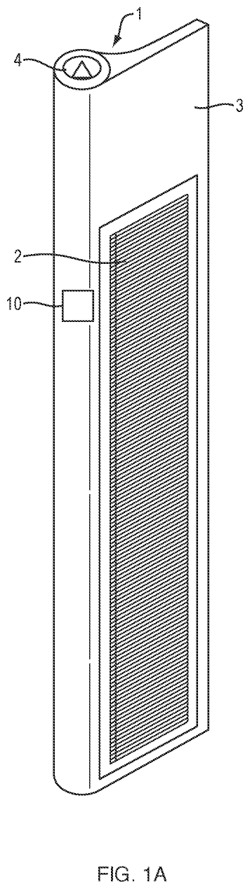

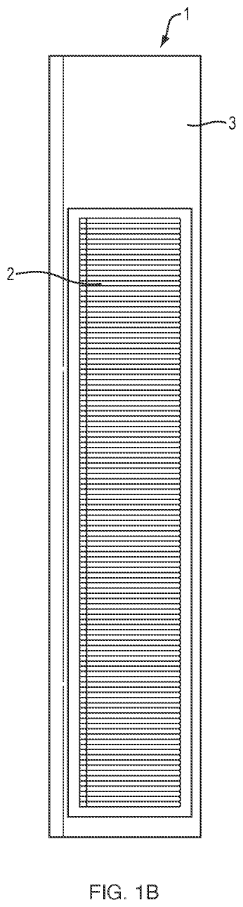

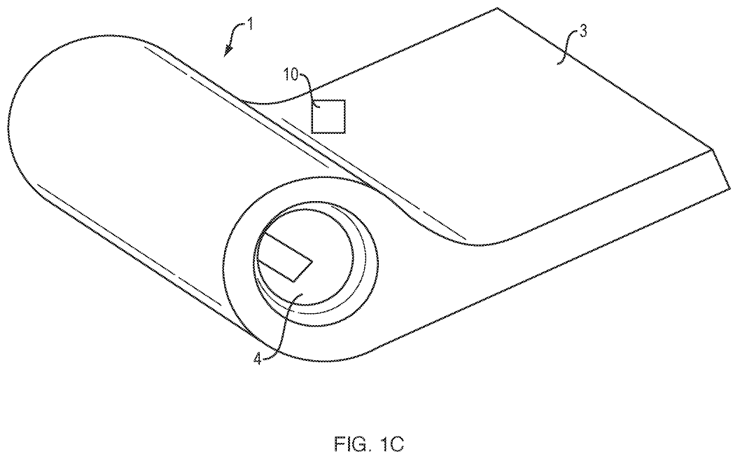

FIG. 1A, FIG. 1B, and FIG. 1C provide side, front, and top views of some embodiments of a TFF assembly 1 designed to function within a bioreactor. The TFF assembly 1 includes a microporous membrane 2 supported by a baffle 3. The TFF assembly 1 is rotatably or pivotally attached to the bioreactor at a movable attachment point 4. The movable attachment point 4 allows the TFF assembly 1 to pivot or rotate when contacted with fluid in the bioreactor. In some embodiments, the baffle 3 pivots less than 360° around the movable attachment point 4. In some embodiments, the TFF assembly 1 also includes a collection receptacle 10 to capture the target product, waste material, or impurity removed from the fluid in the bioreactor during bioprocessing.

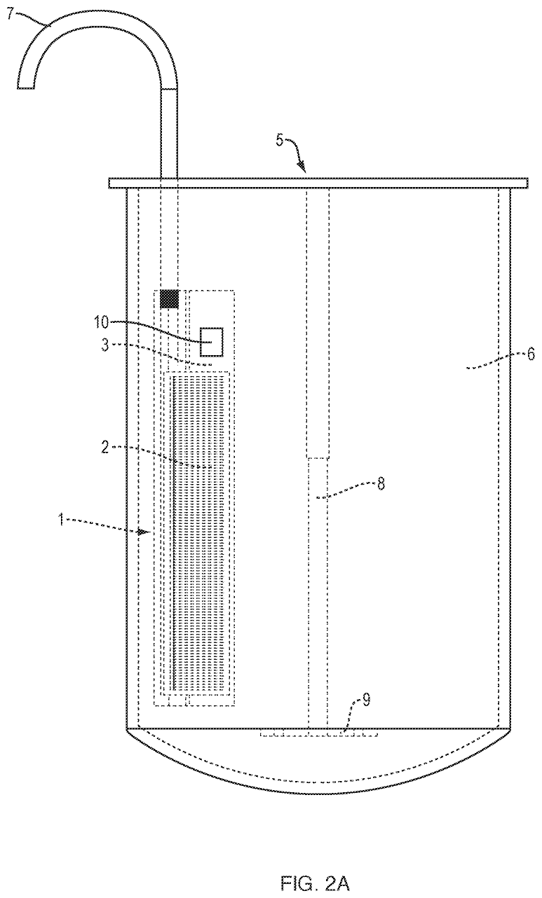

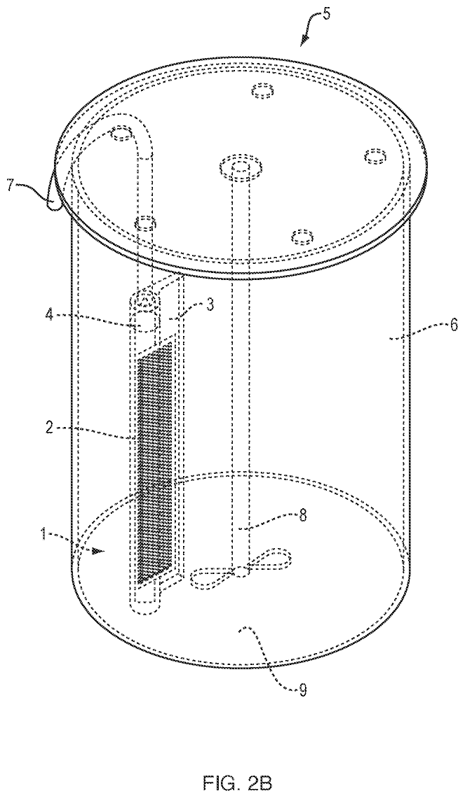

FIG. 2A and FIG. 2B provide side and upper perspective views of some embodiments of a bioreactor 5 disclosed herein. The bioreactor 5 includes a sealed inner volume 6 encompassing the TFF assembly 1.

More than one TFF assembly 1 may be included within the inner volume 6. The bioreactor 5 may be a disposable container made of weldable plastic, such as polyethylene, or the bioreactor 5 may be metal or glass. In some embodiments, the bioreactor 5 has a minimum working volume of 0.5 L and a maximum working volume of 1000 L.

The TFF assembly 1 is attached to an outlet 7, which may be made of glass, metal, or plastic material. The outlet 7 may be attached to an external pump to remove waste materials, impurities, or target product from the bioreactor 5. The attachment of the components of the bioreactor is fluid tight, for example, by welding or gluing.

The bioreactor 5 further includes a mixer 8 within the inner volume 6 to drive cell growth by enabling contact between cells and fresh cell culture media. The mixer 8 includes one or more blades 9. The number and shape of the blades 9 is not particularly limited, provided they provide sufficient agitation of the fluid within the inner volume 6 when actuated. The mixer 8 may be constructed of plastic material, such as polyethylene, or any polymer resistant to gamma irradiation, such as a polypropylene co-polymer. In some embodiments, at least a portion of the mixer 8 is within the inner volume 6, and a mechanical driver for the mixer 8 may be external to the inner volume 6. In some embodiments, the mixer 8 is positioned at or near the bottom of the inner volume 6, when the bioreactor 5 is in mixing position. For a single-use or disposable bioreactor, mixing position may be a hanging position.

When the mixer 8 is rotating during perfusion cell culture, vortices frequently occur, which negatively affect the uniformity of the contents of the bioreactor 5. The particular dimensions of the baffle 3 depend at least in part on the size of the inner volume 6. In some embodiments, the baffle 3 is placed in the inner volume 6, such that it extends through the vortex (or the region where the vortex would form in the absence of the baffle 3) at some level.

The TFF assembly 1 achieves filtration by the flow of the contents of the bioreactor 5, while the mixer 8 is rotating, over the microporous membrane 2. In some embodiments, the movable attachment point 4 allows the TFF assembly 1 to rotate or pivot at least 90° compared to a reference point within the bioreactor. In some embodiments, the movable attachment point 4 allows the TFF assembly 1 to rotate or pivot at least 180° compared to a reference point within the bioreactor. In some embodiments, the movable attachment point 4 is attached to the surface enclosing the inner volume 6.

### IV. Definitions

Unless defined otherwise, all technical and scientific terms used herein have the same meaning as commonly understood by one of ordinary skill in the art to which this invention pertains.

As used herein, the singular forms “a”, “an,” and “the” include plural unless the context clearly dictates otherwise.

The term “bioprocessing,” as used herein, refers to any application of the biological systems of living cells or their components, such as bacteria, enzymes, or chloroplasts, to obtain a target product. In some embodiments, bioprocessing takes place in a biocontainer, such as a bioreactor. Bioprocessing may encompass upstream and downstream bioprocessing. Upstream bioprocessing includes cell culture.

The term “bioreactor,” “biocontainer,” or “fermenter,” as used herein, refers to any manufactured or engineered device or system that supports a biologically active environment. In some instances, a bioreactor is a vessel in which a cell culture process is carried out, which involves organisms or biochemically active substances derived from such organisms. Such a process may be aerobic or anaerobic. Commonly used bioreactors are typically cylindrical, ranging in size from liters to cubic meters, and are often made of stainless steel. In some embodiments described herein, a bioreactor is made of a material other than steel and is disposable or single-use. It is contemplated that the total volume of a bioreactor may be any volume ranging from 100 mL to up to 10,000 liters or more, depending on the process.

The term “cell culture,” as used herein, refers to cells grown in suspension, roller bottles, flasks, and the like, as well as the components of the suspension itself, including, but not limited to cells, cell debris, cellular contaminants, colloidal particles, biomolecules, host cell proteins (HCP), and deoxyribonucleic acid (DNA), mAbs, and flocculants. Large scale approaches, such as bioreactors, including adherent cells growing attached to microcarriers in stirred fermenters, are also encompassed by the term “cell culture.” Moreover, it is possible to not only to culture contact-dependent cells, but also to use the suspension culture techniques in the methods of the claimed invention. Exemplary microcarriers include, for example, dextran, collagen, plastic, gelatin, or cellulose. Porous carriers, such as, for example, Cytoline® or Cytopore®, as well as dextran-based carriers, such as DEAE-dextran (Cytodex 1®), quaternary amine-coated dextran (Cytodex® 2) or gelatin-based carriers, such as gelatin-coated dextran (Cytodex® 3) may also be used. Cell culture procedures for both large and small-scale production of proteins are encompassed by the present invention. Procedures including, but not limited to, a fluidized bed bioreactor, hollow fiber bioreactor, roller bottle culture, or stirred tank bioreactor system may be used, with or without microcarriers, and operated alternatively in a batch, fed-batch, or perfusion mode.

The terms “cell culture medium” and “culture medium,” as used herein, refer to a nutrient solution used for growing animal cells, e.g., mammalian cells. Such a nutrient solution generally includes various factors necessary for cell attachment, growth, and maintenance of the cellular environment. For example, a typical nutrient solution may include a basal media formulation, various supplements depending on the cell type and, occasionally, antibiotics. In some embodiments, a nutrient solution may include at least one component from one or more of the following categories: 1) an energy source, usually in the form of a carbohydrate such as glucose; 2) all essential amino acids, and usually the basic set of twenty amino acids plus cystine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The nutrient solution may optionally be supplemented with one or more components from any of the following categories: 1) hormones and other growth factors as, for example, insulin, transferrin, and epidermal growth factor; 2) salts and buffers as, for example, calcium, magnesium, and phosphate; 3) nucleosides and bases such as, for example, adenosine and thymidine, hypoxanthine; and 4) protein and tissue hydrolysates. In general, any suitable cell culture medium may be used. The medium may be comprised of serum, e.g. fetal bovine serum, calf serum or the like. Alternatively, the medium may be serum free, animal free, or protein free.

The terms “contaminant,” “impurity,” “waste,” and “debris,” as used interchangeably herein, refer to any foreign or objectionable material, including a biological macromolecule, such as DNA, RNA, one or more host cell proteins (HCPs or CHOPs), endotoxins, viruses, lipids and one or more additives which may be present in a sample containing a protein or polypeptide of interest (e.g., an antibody) being separated from one or more of the foreign or objectionable molecules using a stimulus responsive polymer according to the present invention. In some embodiments, a stimulus responsive polymer described herein binds and precipitates a protein or polypeptide of interest from a sample containing the protein or polypeptide of interest and one or more impurities.

The term “continuous process,” as used herein, refers to a process for purifying a target molecule, which includes two or more process steps (or unit operations), such that the output from one process step flows directly into the next process step in the process, without interruption, and where two or more process steps can be performed concurrently for at least a portion of their duration. In other words, in case of a continuous process, as described herein, it is not necessary to complete a process step before the next process step is started, but a portion of the sample is always moving through the process steps. The term “continuous process” also applies to steps within a process step, in which case, during the performance of a process step including multiple steps, the sample flows continuously through the multiple steps that are necessary to perform the process step. One example of such a process step described herein is the flow through purification step which includes multiple steps that are performed in a continuous manner, e.g., flow-through activated carbon followed by flow-through AEX media followed by flow-through CEX media followed by flow-through virus filtration.

The terms “TFF assembly,” “TFF system,” and “TFF apparatus,” as used herein, are interchangeable to refer to a tangential flow filtration (TFF) system that is configured for operation in a single-pass mode and/or a recirculation mode (e.g., full or partial recirculation) and/or alternating flow mode.

The terms “microfiltration membrane,” “MF membrane,” or “microporous membrane,” as used herein, refer to membranes that have pore sizes in the range between about 0.1 μm to about 10 μm capable of use in a filtration system, such as a TFF system.

The terms “product” and “target product,” as used herein, refers to a target compound or target molecule produced in a bioreactor during cell culture. Typically, a product will be a biomolecule (e.g., protein) of interest produced by cell culture.

The terms “purifying,” “purification,” “filtering,” “separate,” “separating,” “separation,” “isolate,” “isolating,” or “isolation,” as used herein, refer to increasing the degree of purity of a target product from a sample comprising the target product and one or more impurities. Typically, the degree of purity of the target product is increased by removing (completely or partially) at least one impurity from the sample.

The term “therapeutic modality” as used herein refers to a target product to treat or prevent a disease, disorder, or condition known in the art.

### EQUIVALENTS

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described is included some embodiments of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A tangential flow filtration (TFF) assembly comprising:
a microporous membrane; and
a baffle supporting the microporous membrane.

2. The tangential flow filtration (TFF) assembly of claim 1, wherein the baffle rotates or pivots about a point.

3. The tangential flow filtration (TFF) assembly of claim 1, wherein the baffle is a flat plate.

4. The tangential flow filtration (TFF) assembly of claim 1, wherein the baffle is shaped as at least one shape selected from the group consisting of: a rectangle, a trapezoid, a parallelogram, a circle, an ellipse, a racetrack, a triangle, and a ladder.

5. The tangential flow filtration (TFF) assembly of claim 1, further comprising a collection receptacle sealed to the baffle.

6. The tangential flow filtration (TFF) assembly of claim 5, further comprising an outlet on the collection receptacle.

7. A bioreactor comprising:
an inner volume enclosed by at least one side wall;
a tangential flow filtration (TFF) assembly including a baffle supporting a microporous membrane, wherein the filtration assembly is movably attached to the side wall; and
a mixer within the inner volume.

8. The bioreactor of claim 7, further comprising one or more inlets or one or more outlets.

9. The bioreactor of claim 7, wherein the baffle spans the height of the inner volume to disrupt vortex formation.

10. The bioreactor of claim 7, wherein the baffle spans enough of the radial dimension of the inner volume to disrupt vortex formation.

11. The bioreactor of claim 7, wherein the bioreactor is single use and/or disposable.

12. The bioreactor of claim 7, wherein the baffle is a flat plate.

13. The bioreactor of claim 7, wherein the baffle is shaped as at least one shape selected from the group consisting of: a rectangle, a trapezoid, a parallelogram, a circle, an ellipse, a racetrack, a triangle, and a ladder.

14. The bioreactor of claim 7, further comprising more than one tangential flow filtration (TFF) assembly.

15. The bioreactor of claim 7, wherein the bioreactor is a perfusion bioreactor.

16. The bioreactor of claim 7, wherein the bioreactor comprises a flexible material.

17. The bioreactor of claim 7, wherein the bioreactor is collapsible.

18. The bioreactor of claim 7, wherein the tangential flow filtration (TFF) assembly is attached to the side wall.

19. The bioreactor of claim 7, further comprising a collection receptacle sealed to the baffle.

20. The bioreactor of claim 19, further comprising an outlet on the collection receptacle.

21. The bioreactor of claim 20, wherein the collection receptacle does not include an outlet.

22. The bioreactor of claim 7, wherein the bioreactor does not include an external pump.

23. The bioreactor of claim 7, wherein the bioreactor does not include a feed line.

24. The bioreactor of claim 7, wherein the bioreactor is self-contained.

25. The bioreactor of claim 7, further comprising an external pump.

26-35. (canceled)
